(19) United States
(12) Patent Application Publication
(10) Pub. No.: US 2025/0002675 A1
(43) Pub. Date: Jan. 2, 2025

(54) PRODUCTION OF VIRGIN-QUALITY PET AND COPOLYESTER RAW MATERIALS FROM POLYESTER CARPET FIBERS

(71) Applicant: Eastman Chemical Company, Kingsport, TN (US)

(72) Inventors: Anne-Martine Sherbeck Jackson, Kingsport, TN (US); Travis Wynn Keever, Jonesborough, TN (US); Michael Paul Ekart, Kingsport, TN (US)

(73) Assignee: Eastman Chemical Company, Kingsport, TN (US)

(21) Appl. No.: 18/697,882

(22) PCT Filed: Oct. 4, 2022
(86) PCT No.: PCT/US2022/045598

§ 371 Date: Apr. 2, 2024

Related U.S. Application Data
(60) Provisional application No. 63/262,152, filed on Oct. 6, 2021.

(51) Int. Cl. C08J 11/24

(52) U.S. Cl. CPC C08J 11/24; C08J 2367/03

(57) ABSTRACT

A process for chemically recycling waste polyethylene terephthalate (PET) carpet fibers. The process comprises providing a waste carpet fiber composition comprising at least 75 wt % of PET and 6 wt % or less of ash; reacting the waste carpet fiber composition with methanol to produce a depolymerized poly ester mixture comprising polyester oligomers, dimethyl terephthalate (DMT), and ethylene glycol (EG); and recovering the DMT and the EG from the depolymerized polyester mixture.

### FIELD OF THE INVENTION

The invention generally relates to the chemical recycling of polyester carpet fibers using methanol.

### BACKGROUND OF THE INVENTION

National and global efforts are focusing on reusing and recycling post-consumer and post-industrial materials, specifically polyethylene terephthalate (PET) and PET-like materials (any polymer material containing a high concentration of terephthalate (TPA)). This includes a range of materials used in a wide range of applications, such as drink bottles, packaging, textiles, carpets, thermoforms, multi-layer films, and plasticizers.

Some of the more conventional and less complex materials can be recycled through simple mechanical recycling processes. However, due to the large and ever-growing brand commitments and legislation around using sustainable or recycle-containing materials, alternative feedstocks that are not compatible with the existing mechanical recycling streams must be considered to create a viable circular economy.

One of those feedstocks is polyester carpet fibers. Composed of PET, heavy colorants, stain guards, polypropylene, inorganic compounds (e.g., TiO₂), and other additives; polyester carpet fibers pose a particularly difficult challenge for reproducing PET raw materials because of the difficulty in getting the proper purification.

Thus, there is a need to provide alternative and/or improved methods for producing virgin-quality PET raw materials, which can be used in polymer production, from polyester carpet fibers.

The present invention addresses this need as well as others, which will become apparent from the following description and the appended claims.

### SUMMARY OF THE INVENTION

The invention is as set forth in the appended claims.

Briefly, in one aspect, the invention provides a process for chemically recycling waste polyethylene terephthalate (PET) carpet fibers. The process comprises:
providing a waste carpet fiber composition comprising at least 75 wt % of PET and 6 wt % or less of ash;
reacting the waste carpet fiber composition with methanol to produce a depolymerized polyester mixture comprising polyester oligomers, dimethyl terephthalate (DMT), and ethylene glycol (EG); and
recovering the DMT and the EG from the depolymerized polyester mixture,
wherein the weight percentages are based on the total weight of the waste carpet fiber composition.

In another aspect, the invention provides a mixture for chemical recycling of waste carpet fibers. The mixture comprises the reaction product of:
(a) a waste carpet fiber composition comprising at least 75 wt % of polyethylene terephthalate (PET) and 6 wt % or less of ash; and
(b) methanol,
wherein the weight percentages are based on the total weight of the waste carpet fiber composition.

In yet another aspect, the invention provides a process for preparing a recycled polyester. In one variation, the process comprises:
using the purified EG or DMT or both obtained according to the invention to prepare a recycled polyester.

In another variation, the process comprises:
reacting the purified DMT obtained according to the invention with water to form a recycled terephthalic acid (rTPA); and
using the rTPA and optionally the purified EG also obtained according to the invention to prepare a recycled polyester.

In yet another variation, the process comprises:
reacting the purified DMT obtained according to the invention with virgin EG, the purified EG also obtained according to the invention, or both to form bis(2-hydroxyethyl) terephthalate (BHET) or oligomers thereof; and
polycondensing the BHET or the oligomers thereof to form recycled PET.

### BRIEF DESCRIPTION OF THE DRAWINGS

FIG. 1 is a flow diagram of an exemplary laboratory methanolysis process used in the working examples.

FIG. 2 is a graph of rate data from the methanolysis of several different polyester carpet fiber samples from Example 2.

FIG. 3 is a graph of rate data from the methanolysis of various mixtures of two polyester carpet fiber samples from Example 2.

FIG. 4 is a graph comparing the median MeOH/DMT values of the Control and A-D samples from Example 2 with the feedstock characteristic, PET %.

FIG. 5 is a graph comparing the median MeOH/DMT values of the Control and A-D samples from Example 2 with the feedstock characteristic, Ash %.

### DETAILED DESCRIPTION OF THE INVENTION

It has been surprisingly discovered that virgin-quality PET raw materials, which can be used in polymer production, can be obtained from waste polyester carpet fiber. In various embodiments, mechanical separation, depolymerization, and extensive purification technologies are employed to produce virgin-like PET raw materials from waste polyester carpet fiber. The mechanical separation processes can include shredding, sorting, sink/float, grinding, pulverization, granulation, and others, depending on where the carpet fiber enters the feed stream. After the mechanical processes, a PET depolymerization process uses methanol and optionally ester exchange catalyst(s) to break the PET down into purifiable building blocks. The purification technologies utilize physical properties, such as boiling point, solubility, diffusion coefficient, density, surface tension, and particle size in processes such as filtration, centrifugation, and distillation, to remove the impurities or contaminants in the complex multi-component polyester materials. The combination of these processes can remove items normally found in commercial carpets, such as colorants, stain guards, polypropylene, and inorganic compounds. The breakdown and purification of such carpets into individual PET building blocks are described below.

As used herein, “PET” or “polyethylene terephthalate” refers to a homopolymer of polyethylene terephthalate, or to a polyethylene terephthalate modified with one or more acid and/or glycol modifiers and/or containing residues or moieties of other than ethylene glycol and terephthalic acid, such as isophthalic acid, 1,4-cyclohexanedicarboxylic acid, diethylene glycol, 2,2,4,4-tetramethyl-1,3-cyclobutanediol (TMCD), cyclohexanedimethanol (CHDM), propylene glycol, isosorbide, 1,4-butanediol, 1,3-propane diol, and/or neopentylglycol (NPG). Also included within the definition of the terms “PET” and “polyethylene terephthalate” are polyesters having repeating terephthalate units (whether or not they contain repeating ethylene glycol based units) and one or more residues or moieties of a glycol including, for example, TMCD, CHDM, propylene glycol, or NPG, isosorbide, 1,4-butanediol, 1,3-propane diol, and/or diethylene glycol, or combinations thereof. Examples of polymers with repeat terephthalate units can include, but are not limited to, polypropylene terephthalate, polybutylene terephthalate, and copolyesters thereof.

In one aspect, the invention provides a process for chemically recycling waste polyethylene terephthalate (PET) carpet fibers. The process comprises:
providing a waste carpet fiber composition comprising at least 75 wt % of PET and 6 wt % or less of ash;
reacting the waste carpet fiber composition with methanol (optionally in the presence of an ester exchange catalyst) to produce a depolymerized polyester mixture comprising polyester oligomers, dimethyl terephthalate (DMT), and ethylene glycol (EG); and
recovering the DMT and the EG from the depolymerized polyester mixture,
wherein the weight percentages are based on the total weight of the waste carpet fiber composition.

### Carpet Fiber Feedstocks

There are two potential sources of PET carpet fiber feedstocks: post-industrial carpet fibers and post-consumer carpet fibers. Post-industrial carpet fibers are recovered from their manufacturing process prior to tufting, in which the PET carpet fibers are attached to the carpet backing. Post-consumer carpet fibers, on the other hand, are removed from the carpet backing by a variety of processes to separate the PET fibers from the other carpet components.

Impurities in post-industrial PET carpet fibers can prevent their direct use in mechanical recycling processes outside of the textile industry. These impurities can also pose a challenge for the methanolysis process and for the purification of the recovered monomers, DMT and EG. A few of the contaminants present in post-industrial carpet are listed below:
1. Color bodies and dyes intentionally added to the polyester;
2. Dimethyl Isophthalate (DMI)—often used in PET manufacturing;
3. Diethylene glycol (DEG)—often a byproduct of PET manufacturing;
4. Factory applied stain guards (e.g., perfluorochemicals); and
5. TiO₂—in carpet fibers for opaque luster.

Recycling post-consumer carpet poses additional challenges beyond those of post-industrial carpet fibers. As with all post-consumer recycled material, there is an inherent variability due to the material being aggregated from different manufacturers, produced at different times, and used in different areas of the country. The aggregation process also introduces the potential for cross-contamination from other carpet types, such as nylon or polytrimethylene terephthalate (PTT) carpets. There is the added complication of needing to remove the PET face fiber from the carpet backing, which can introduce contamination from the non-fiber portions of the carpet, such as CaCO₃, polypropylene (PP), and adhesives. To add to the variability of this feedstock, there are additional contaminants that have been introduced during the lifetime of the carpet, such as cleaners, salts, sand, dirt, and other waste materials. The variability and contamination in post-consumer carpet fibers can render this material unusable for all types of mechanical recycling and pose a challenge for methanolysis and monomer purification. A list of contaminants that can be present in post-consumer carpet fibers is provided below:
1. Cleaning agents (e.g., surfactants, solvents), etc.;
2. Consumer applied stain guards (e.g., silicones);
3. Professionally applied stain guards (e.g., perfluorochemicals);
4. Road salts (e.g., NaCl, MgCl₂);
5. Sand (e.g., SiO₂);
6. Dirt;
7. Human skin, hair, and fluids;
8. Pet urine and feces;
9. Food waste;
10. Nylon 6 or Nylon 66 and monomeric components;
11. PTT and monomeric components (e.g., 1,3-propanediol);
12. Polypropylene (carpet backing material);
13. CaCO₃ (carpet backing material); and
14. Adhesives (e.g., styrene-butadiene rubber, low-melt PET, polyvinyl butyral (PVB), etc.).

The waste carpet fiber composition useful in the process of the invention may comprise post-consumer carpet fibers, post-industrial carpet fibers, or both.

In various embodiments, the waste carpet fiber composition may comprise at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-consumer carpet fibers, based on the total weight of the waste carpet fiber composition.

In various embodiments, the waste carpet fiber composition may comprise at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-industrial carpet fibers, based on the total weight of the waste carpet fiber composition.

The waste carpet fiber composition useful in the process of the invention typically comprises at least 75 wt %, at least 80 wt %, at least 85 wt %, at least 90 wt %, at least 95 wt %, at least 99 wt %, or 100 wt % of PET, based on the total weight of the composition.

In the case the waste carpet fiber composition comprises post-consumer carpet fibers, the PET content of the waste carpet fiber composition may desirably be at least 75 wt %, at least 80 wt %, at least 85 wt %, at least 90 wt %, at least 95 wt %, at least 99 wt %, or 100 wt %, based on the total weight of the composition.

In the case the waste carpet fiber composition comprises post-industrial carpet fibers, the PET content of the waste carpet fiber composition may desirably be at least 90 wt %, at least 95 wt %, at least 99 wt %, or 100 wt %, based on the total weight of the composition.

The PET content may be calculated using the total dicarboxylic acid content in the waste carpet fiber composition. For example, since PET may contain some amount of isophthalic acid (IPA) residues in addition to terephthalic acid (TPA) residues, the PET content may be calculated based on the total content of TPA and IPA.

The waste carpet fiber composition useful in the process of the invention may comprise up to 6 wt %, up to 5 wt %, up to 4 wt %, up to 3 wt %, up to 2 wt %, up to 1 wt %, up to 0.5 wt %, up to 0.1 wt %, up to 0.01 wt %, or 0 wt % of ash, based on the total weight of the composition.

By “ash”, it is meant the inorganic residues of the waste carpet fiber composition after undergoing a destructive ashing procedure. The ashing procedure involves heating a sample of the waste carpet fiber composition in air at 800° C. for 3 hours. The residues may be from antiblock, fillers, reinforcements, catalysts, colorants, etc. in the waste carpet fiber composition.

In various embodiments, the waste carpet fiber composition may comprise greater than 0 up to 6 wt %, greater than 0 up to 5 wt %, greater than 0 up to 4 wt %, greater than 0 up to 3 wt %, greater than 0 up to 2 wt %, greater than 0 up to 1 wt %, greater than 0 up to 0.5 wt %, greater than 0 up to 0.1 wt %, or greater than 0 up to 0.01 wt % of ash, based on the total weight of the composition.

In the case the waste carpet fiber composition comprises post-consumer carpet fibers, the ash content of the waste carpet fiber composition may desirably be up to 6 wt %, up to 5 wt %, up to 4 wt %, up to 3 wt %, up to 2 wt %, up to 1 wt %, up to 0.5 wt %, up to 0.1 wt %, up to 0.01 wt %, or 0 wt %, based on the total weight of the composition. In each case, the ash content may be greater than 0.

In the case the waste carpet fiber composition comprises post-industrial carpet fibers, the ash content of the waste carpet fiber composition may desirably be up to 1 wt %, up to 0.5 wt %, up to 0.1 wt %, up to 0.01 wt %, or 0 wt %, based on the total weight of the composition. In each case, the ash content may be greater than 0.

The waste carpet fiber composition useful in the process of the invention may comprise up to 10 wt %, up to 3 wt %, or 0 wt % of the residues of isophthalic acid, based on the total weight of the waste carpet fiber composition.

In the case the waste carpet fiber composition comprises post-consumer carpet fibers, the isophthalic acid residues content of the waste carpet fiber composition may desirably be up to 10 wt %, up to 3 wt %, or 0 wt %, based on the total weight of the waste carpet fiber composition.

In the case the waste carpet fiber composition comprises post-industrial carpet fibers, the isophthalic acid residues content of the waste carpet fiber composition may also desirably be up to 10 wt %, up to 3 wt %, or 0 wt %, based on the total weight of the waste carpet fiber composition.

The waste carpet fiber composition useful in the process of the invention may comprise up to 15 wt %, up to 5 wt %, or 0 wt % of the residues of 1,3-propanediol, based on the total weight of the waste carpet fiber composition.

In the case the waste carpet fiber composition comprises post-consumer carpet fibers, the 1,3-propanediol residues content of the waste carpet fiber composition may desirably be up to 15 wt %, up to 5 wt %, or 0 wt %, based on the total weight of the waste carpet fiber composition.

In the case the waste carpet fiber composition comprises post-industrial carpet fibers, the 1,3-propanediol residues content of the waste carpet fiber composition may also desirably be up to 15 wt %, up to 5 wt %, or 0 wt %, based on the total weight of the waste carpet fiber composition.

The waste carpet fiber composition useful in the process of the invention may comprise up to 5000 ppm, up to 1500 ppm, up to 200 ppm, up to 80 ppm, or 0 ppm of nitrogen, based on the total weight of the waste carpet fiber composition.

In the case the waste carpet fiber composition comprises post-consumer carpet fibers, the nitrogen content of the waste carpet fiber composition may desirably be up to 5000 ppm, up to 1500 ppm, up to 200 ppm, up to 80 ppm, or 0 ppm, based on the total weight of the waste carpet fiber composition.

In the case the waste carpet fiber composition comprises post-industrial carpet fibers, the nitrogen content of the waste carpet fiber composition may also desirably be up to 200 ppm, up to 80 ppm, or 0 ppm, based on the total weight of the waste carpet fiber composition.

The waste carpet fiber composition useful in the process of the invention may comprise undensified waste carpet fibers.

Alternatively, the waste carpet fiber composition useful in the process of the invention may comprise densified waste carpet fibers, such as by melt extrusion (e.g., into pellets), molding (e.g., into briquettes), or agglomeration (e.g., through externally applied heat, heat generated by frictional forces, or by adding one or more adherents). As used herein, the term “densified” refers to a material that has undergone one or more processing steps to increase its bulk density to at least 0.20 g/cm³.

In one or more embodiments, the bulk density of the densified waste carpet fibers can be at least 0.22, at least 0.25, at least 0.27, at least 0.30, at least 0.32, or at least 0.35 g/cm³ and/or not more than 0.50, not more than 0.47, not more than 0.45, not more than 0.42, not more than 0.40, or not more than 0.37 g/cm³. The densified waste carpet fibers may have undergone one or more processing steps including, for example, cutting, chopping or other size reduction, separation of two or more different types of components, heating (and optionally melting), and pelletizing or solidifying.

In one or more embodiments, the densified waste carpet fibers can comprise particulates, pellets, granules, chunks, or particles having a D90 particle size of at least 0.1, at least 0.5, at least 1, at least 1.5, at least 2, at least 2.5, at least 3, at least 3.5, at least 4, at least 4.5, or at least 5 mm and/or not more than 10, not more than 8, not more than 6, not more than 5, not more than 3, not more than 2, not more than 1, or not more than 0.5 mm.

In one or more embodiments, the waste carpet fiber composition can include at least 80, at least 85, at least 90, at least 95, at least 98, at least 99, at least 99.5, or at least 99.9 wt % of waste carpet fibers, based on the total weight of the waste carpet fiber composition.

In one or more embodiments, the waste carpet fiber composition can include at least 80, at least 85, at least 90, at least 95, at least 98, at least 99, at least 99.5, or at least 99.9 wt % of densified waste carpet fibers, based on the total weight of the waste carpet fiber composition.

The waste carpet fiber composition useful in the process of the invention may comprise any combination of parameters described herein. For example, the waste carpet fiber composition may comprise any combination of PET and ash content; PET, ash, and/or IPA content; PET, ash, IPA, and/or 1,3-propanediol content; PET, ash, IPA, 1,3-propanediol, and/or nitrogen content described herein.

In various embodiments, the waste carpet fiber composition comprises post-consumer carpet fiber and 75 to 100 wt % of PET, 0 to 10 wt % of IPA residues, 0 to 6 wt % of ash, 0 to 15 wt % of 1,3-propanediol residues, and 0 to 5000 ppm of nitrogen, based on the total weight of the waste carpet fiber composition.

In various other embodiments, the waste carpet fiber composition comprises post-consumer carpet fiber and 90 to 100 wt % of PET, 0 to 3 wt % of IPA residues, 0 to 3 wt % of ash, 0 to 5 wt % of 1,3-propanediol residues, and 0 to 1500 ppm of nitrogen, based on the total weight of the waste carpet fiber composition.

In various embodiments, the waste carpet fiber composition comprises post-industrial carpet fiber and 90 to 100 wt % of PET, 0 to 10 wt % of IPA residues, 0 to 1 wt % of ash, 0 to 15 wt % of 1,3-propanediol residues, and 0 to 200 ppm of nitrogen, based on the total weight of the waste carpet fiber composition.

In various other embodiments, the waste carpet fiber composition comprises post-industrial carpet fiber and 95 to 100 wt % of PET, 0 to 3 wt % of IPA residues, 0 to 0.5 wt % of ash, 0 to 5 wt % of 1,3-propanediol residues, and 0 to 80 ppm of nitrogen, based on the total weight of the waste carpet fiber composition.

The waste carpet fiber compositions useful in the process of the invention may be obtained commercially from waste carpet fiber reclaimers. Alternatively, in the case of post-industrial carpet fibers, they may be obtained directly from carpet manufacturers. In the case of post-consumer carpet fibers, the waste carpet fiber composition may be obtained by removing the fibers from the carpet backing by a variety of techniques, such as shearing, and optionally subjecting the fibers to one or more processing techniques, such as shredding, sorting, sink/float, grinding, pulverization, granulation, etc.

### Methanolysis

After providing a waste carpet fiber composition suitable for chemical recycling, the process of the present invention involves subjecting the composition to methanolysis. During methanolysis, the waste carpet fiber composition is reacted with methanol (optionally in the presence of an ester exchange catalyst) to produce a depolymerized polyester mixture comprising polyester oligomers, dimethyl terephthalate (DMT), and ethylene glycol (EG). Other monomers, such as 1,4-cyclohexanedimethanol (CHDM),

diethylene glycol, dimethyl isophthalate, and 1,3-propane diol, may also be produced, depending on the composition of the waste carpet fiber.

The process of methanolysis has been well documented throughout literature and is effective in producing DMT from PET materials. Some representative examples of the methanolysis of PET include those described in U.S. Pat. Nos. 3,321,510; 3,776,945; 5,051,528; 5,298,530; 5,414,022; 5,432,203; 5,576,456; and 6,262,294; which are incorporated herein by reference. These examples may be used in the process of the present invention.

An example of a suitable methanolysis process can be illustrated with reference to the disclosure of U.S. Pat. No. 5,298,530, which describes a process for the recovery of EG and DMT from scrap polyester (or waste polyester carpet fiber in the case of the present invention). This process includes the steps of dissolving scrap polyester in oligomers of EG and terephthalic acid (TPA) or DMT, and passing superheated methanol through this mixture. The oligomers can comprise any low molecular weight polyester polymer of the same composition as that of the scrap material being employed as the starting component such that the scrap polymer will dissolve in the low molecular weight oligomer. The DMT and the EG are recovered from the methanol vapor stream that issues from the depolymerization reactor.

In the above process, scrap PET can be conveyed through a loading system to a dissolver containing terephthalate oligomers. The loading system can be any conventional system known to persons skilled in the art, such as screw feeders, extruders, or batch adders. The dissolver is equipped with an agitator and means of heating, which could include a jacket, tracing, internal heating coils, and/or an external heat exchanger. At startup, monomers or oligomers of a polyester, such as EG and/or DMT, can be introduced into the dissolver and heated to a temperature of 110° C. to 305° C. For example, the temperature range can be from 230° C. to 290° C. The scrap PET and the oligomers can be agitated in the dissolver for a time sufficient to allow the scrap PET to mix with the oligomers and form a startup melt. Typically, the time needed for mixing can range from 5 minutes to 60 minutes.

The startup melt can be drawn through a strainer and transferred by pump to a depolymerization reactor. Alternatively, all or a portion of the startup melt can be returned to the dissolver, which is useful during startup, as well as after startup should it be desired, to provide molten polyester to the top of the dissolver to initiate melting of fresh polyester scrap feed.

Superheated methanol vapor can then be passed through the contents of the depolymerization reactor, heating the reactor contents to form a melt comprising low molecular weight polyester oligomers, monohydric alcohol-ended oligomers, glycols, and DMT. Conventional systems can be used to heat and supply the methanol to the reactor and to recover the methanol for reuse such as, for example, the methanol supply and recovery loop described in U.S. Pat. No. 5,051,528.

In lieu of, or in addition to, superheated methanol vapor, methanol in liquid form, saturated methanol vapor, and/or supercritical methanol may be introduced into the depolymerization reactor.

Typically, an excess quantity of methanol is passed through the depolymerization reaction mixture. For example, a methanol to PET mass ratio of 1.1:1 to 10:1 may be used.

A portion of the reactor melt can then be transferred from the reactor back to the dissolver where the reactor-melt reacts and equilibrates with the molten scrap polyester chains to shorten the average chain length of the dissolver contents and thereby greatly decrease the viscosity. Accordingly, the oligomers that are initially introduced into the dissolver are typically needed just at startup. After startup, the process of the present invention can be run continuously without having to further introduce external polyester chain-shortening material to the dissolver. The dissolver can be run at atmospheric pressure with little methanol present, thereby greatly decreasing the risk of methanol leakage and increasing process safety. Simple solids handling devices, such as rotary air locks, can be employed since more elaborate sealing devices are not necessary. The viscosity of the melt transferred from the dissolver is sufficiently low to permit the use of inexpensive pumping means, and it enables the reactor to be operated at pressures significantly higher than atmospheric pressure.

The return of reactor-melt from the depolymerization reactor back to the dissolver may be adjusted to a rate that is selected based on the flow rates of material in and out of the dissolver and the desired ratio of molten reactor contents to molten scrap polyester in the dissolver. For example, the ratio may be from 5 to 90 wt % reactor melt-to-scrap polyester. In another example, the ratio may be from 20 to 50 wt % reactor melt-to-scrap polyester. If desired, the recovery step (described below) can be omitted while the reactor-melt is transferred to the dissolver, for example, during standby operations when there is an interruption in the supply of scrap polyester to the dissolver, during plant startup, or while the melt in the dissolver is brought up to operating levels.

The depolymerization reactor can be run at a higher pressure than the dissolver, eliminating the need for pumping means to transfer the reactor-melt from the reactor to the dissolver. Supplementary pumping means can optionally be provided, if desired. The operating pressure of the depolymerization reactor can be from 0 kPa gauge (0 psig) to 689.5 kPa gauge (100 psig). The reactor is typically operated at a pressure of 206.8 kPa gauge (30 psig) to 344.7 kPa gauge (50 psig).

The temperature of the melt in the depolymerization reactor is typically maintained above the boiling point of methanol at the pressure present in the reactor or above its critical temperature (about 239° C.), to maintain the methanol in the vapor state and to allow it to readily exit from the reactor. For example, the melt temperature in the depolymerization reactor can be from 100° C. to 320° C., from 180° C. to 305° C., or from 250° C. to 290° C.

To facilitate depolymerization, there can be added to the dissolver and/or the reactor an ester exchange catalyst, such as zinc, titanium, manganese, lithium, potassium, and/or magnesium. In various embodiments, the ester exchange catalyst may be a mixture of two or more catalyst metals. The catalyst metals may be introduced in their salt form with anions such as acetates, carbonates, hydroxides, oxides (particularly soluble oxides), methoxides, fluorides, chlorides, bromides, iodides, phosphates, sulfates, nitrates, etc.

In one or more embodiments, the ester exchange catalyst may be zinc acetate, titanium (IV) isopropoxide, lithium acetate, manganese (II) acetate, magnesium methoxide, and/or potassium carbonate.

The catalyst can be employed in a range of 0 to 800 parts by weight of catalyst metal per million parts by weight of solid polyester introduced into the dissolver/reactor. Other catalyst amounts can include 30 to 300 ppm or 30 to 100 ppm.

In one or more other embodiments, the ester exchange catalyst excludes tin, zinc, and/or titanium. In various embodiments, the amount of tin, zinc, and/or titanium in the depolymerization reactor melt is no more than 200, no more than 150, no more than 100, no more than 50, no more than 25, no more than 10 ppm, or no more than 1 ppm by weight of solid polyester introduced into the dissolver/reactor.

The ester exchange catalyst may also be employed with a co-catalyst, such as sodium hydroxide. The co-catalyst can be employed in a range of 0 to 800 parts co-catalyst metal by weight per million parts by weight of solid polyester introduced into the dissolver/reactor. Other co-catalyst amounts can include 30 to 300 ppm or 30 to 100 ppm.

To further facilitate depolymerization, a glycoxide or methoxide may be added to the reaction mixture. The glycoxide or methoxide, which comprises a glycoxide or methoxide anion and a cation, may be selected from an alkali metal glycoxide or methoxide, an alkaline earth metal glycoxide or methoxide, a metal glycoxide or methoxide, an ammonium glycoxide or methoxide, or combinations thereof. Examples of the cation include lithium, sodium, potassium, magnesium, calcium, strontium, barium, zinc, aluminum, and ammonium. In various embodiments, the glycoxide or methoxide may be sodium glycoxide or methoxide, such as monosodium glycoxide. The glycoxide or methoxide may be generated by adding an alkali metal, an alkaline earth metal, or a metal to mono ethylene glycol (MEG). In various embodiments, the glycoxide may be generated by adding sodium hydroxide to MEG, or the methoxide may be generated by adding sodium hydroxide to methanol.

In various embodiments, the molar ratio of glycoxide or methoxide to methanol can range from 0.05:1 to 0.5:1, such as about 0.2:1.

In various embodiments, the molar ratio of glycoxide or methoxide to PET can range from 1:2 to 1:20, or from 1:10 and 1:15.

In one or more embodiments, the average residence time of the waste carpet fiber composition in the reaction zone can be at least 1, 2, 5, 10, or 15 minutes and/or not more than 12, 11, 10, 9, 8, 7, 6, 5, or 4 hours.

In one or more embodiments, at least 50, 55, 60, 65, 70, 75, 80, 85, 90, 95, or 99 wt % of the total weight of PET introduced into the methanolysis reaction zone is decomposed upon leaving the zone.

In one or more embodiments, a reactor purge stream may be removed, either continuously or periodically, from the reaction zone. The reactor purge stream may have a boiling point higher than the boiling point of DMT.

In one or more embodiments, the reactor purge stream may comprise at least 25, at least 30, at least 35, at least 40, at least 45, at least 50, at least 55, at least 60, at least 65, at least 70, at least 75, at least 80, at least 85, at least 90, at least 95, or at least 99 wt % of DMT, based on the total weight of the stream. In one or more embodiments, the reactor purge stream can comprise not more than 25, not more than 20, not more than 15, not more than 10, not more than 5, not more than 2, or not more than 1 wt % of components with a boiling point higher than the boiling point of DMT. Additionally, or alternatively, the reactor purge stream can have a melting temperature of at least 5, at least 10, at least 15, at least 20, or at least 25 and/or not more than 50, not more than 45, not more than 40, not more than 35, not more than 30, not more than 25, not more than 20, or not more than 15° C. lower than the temperature of the reactor.

The reactor purge stream may include at least 100 ppm and not more than 25 wt % of one or more non-DMT solids, based on the total weight of the stream. In one or more embodiments, the total amount of non-DMT solids in the reactor purge stream can be at least 150, at least 200, at least 250, at least 300, at least 350, at least 400, at least 500, at least 600, at least 700, at least 800, at least 900, at least 1000, at least 1500, at least 2000, at least 2500, at least 3000, at least 3500, at least 4000, at least 4500, at least 5000, at least 5500, at least 6000, at least 7000, at least 8000, at least 9000, at least 10,000, or at least 12,500 ppm and/or not more than 25, not more than 22, not more than 20, not more than 18, not more than 15, not more than 12, not more than 10, not more than 8, not more than 5, not more than 3, not more than 2, or not more than 1 wt %, based on the total weight of the stream.

In one or more embodiments, the reactor purge stream has a total solids content of at least at least 100, at least 250, at least 500, at least 750, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500 ppm by weight or at least 1, at least 2, at least 5, at least 8, at least 10, or at least 12 wt % and/or not more than 25, not more than 22, not more than 20, not more than 17, not more than 15, not more than 12, not more than 10, not more than 8, not more than 6, not more than 5, not more than 3, not more than 2, or not more than 1 wt % or not more than 7500, not more than 5000, or not more than 2500 ppm by weight, based on the total weight of the stream.

Examples of non-DMT solids can include, but are not limited to, non-volatile catalyst compounds. In one or more embodiments, the reactor purge stream can include at least 100, at least 250, at least 500, at least 750, at least 1000, at least 1500, at least 2000, at least 2500, at least 3000, at least 3500, at least 4000, at least 4500, at least 5000, at least 7500, at least 10,000, or at least 12,500 ppm and/or not more than 60,000, not more than 50,000, not more than 40,000, not more than 35,000, not more than 30,000, not more than 25,000, not more than 20,000, not more than 15,000, or not more than 10,000 ppm of non-volatile catalyst metals. Examples of non-volatile catalyst metals can include, but are not limited to, titanium, zinc, manganese, methoxide compounds, alkali metals, alkaline earth metals, tin, residual esterification or ester exchange catalysts, residual polycondensation catalysts, aluminum, depolymerization catalysts, and combinations thereof.

A vapor stream comprising DMT, EG, and methanol can be withdrawn from the depolymerization reactor. Depending on the composition of the polyester carpet fiber, other monomers (such as diethylene glycol, triethylene glycol, dimethyl isophthalate, 1,4-cyclohexanedimethanol, 1,3-propane diol, and/or methylhydroxyethyl terephthalate) may also be present in the methanol vapor stream. In addition to being a depolymerization reactant, the methanol vapor aids in removing other vapors from the reactor by acting as a carrier gas stream and by stripping the other gases from the solution. The effectiveness of the superheated methanol for heating the reactor contents and for stripping gases depends on its volumetric flow rate. The depolymerization rate in the reactor, therefore, depends on the methanol flow rate to the reactor. The methanol vapor stream exiting the depolymerization reactor may be passed to a distillation device to separate most of the methylhydroxyethyl terephthalate from the vapor stream. The recovered methylhydroxyethyl terephthalate may be passed to the dissolver and/or the reactor where it is useful as a low molecular weight oligomer for shortening the average polyester chain length and decreasing the viscosity of the melt in the dissolver/reactor.

The vapor stream may then be transferred to a second distillation device which separates methanol from the other vapor stream components. The methanol can be recovered for further use as described in U.S. Pat. No. 5,051,528 (incorporated herein by reference). The remaining recovered vapor stream components can be transferred to other separation devices, e.g., distillation columns and crystallizers, where the DMT, EG, and, optionally, other monomers can be separated out.

The methanolysis process may be carried out as a semi-continuous or continuous process. After initial startup, the startup oligomers described above do not have to be provided from a source external to the process; that is, the melt provided from the depolymerization reactor and/or the methylhydroxyethyl terephthalate provided from optional distillation of the methanol vapor stream, to the dissolver can shorten the average polyester chain length and sufficiently decrease the melt viscosity in the dissolver.

Most of the contaminants in the scrap or waste PET carpet fiber composition can be removed from the melt in the dissolver before introducing the melt to the depolymerization reactor. For example, inorganic contaminants such as metals or sand can be removed by straining the melt from the dissolver. Polyolefins and other contaminants, such as polyethylene, polystyrene, and polypropylene, tend to float on top of the melt in the dissolver and can be drawn off to a separator, removed, and the polyolefin-free melt can be returned to the dissolver. Soluble contaminants can be allowed to accumulate in the melt in the dissolver and can be routinely purged with oligomers from the depolymerization reactor. Alternatively, they can be removed from the melt flowing from the reactor back to the dissolver.

In addition to or in combination with the waste carpet fiber composition, the methanolysis reacting step according to the present invention can accept a broad array of other PET-containing waste products, such as textiles, bottle flake, reclaimer waste, or combinations thereof, to produce recycled monomer feedstocks for repolymerization into polyesters.

The rate at which the methanolysis reaction proceeds can be assessed by calculating the molar ratio of the MeOH consumed to the DMT produced over time (MeOH/DMT molar ratio). Lower values indicate that less methanol was used to produce a mole of DMT and are, therefore, more efficient and desirable. In various embodiments, the reacting step of the invention can provide a MeOH/DMT molar ratio of 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, or 4 or less throughout the duration of the reaction. The denominator in the MeOH/DMT molar ratio includes DMT derivatives, such as methylhydroxyethyl terephthalate (MHET).

### Recovery and Purification

The DMT and EG in the depolymerized polyester mixture produced during methanolysis reaction can be recovered and purified by crystallization, filtration, distillation, or a combination of these. Additional techniques for recovering and purifying the monomer(s) include adsorption (e.g., with activated carbon, charcoal, silica gel, etc.), anion or cation exchange, and/or liquid extraction. Other monomers that may be present in the waste carpet fiber composition, such as dimethyl isophthalate, 1,4-cyclohexanedimethanol, dimethyl 1,4-cyclohexanedicarboxylate, 1,3-propane diol, and diethylene glycol, can also be recovered by the techniques noted above, and repolymerized into polyesters.

In various embodiments, the methanol vapor stream exiting from the depolymerization reactor can comprise a gas-phase stream comprising DMT, EG, methanol, and small amounts of impurities. The amount of impurities in the methanol vapor stream depends on the relative volatility of the impurities and DMT. If the volatility of the impurities is low enough, some of the impurities will be carried out of the reactor in substantial concentrations.

In various embodiments, the methanol vapor stream can be cooled and condensed to form a condensate comprising DMT dissolved in methanol. The temperature of this stream is then reduced and some of the methanol removed, causing the dissolved DMT to precipitate as crystals. The solids can then be separated by an appropriate separation method, such as filtration and/or centrifugation. The crystals can then be washed to remove most of the EG and other contaminants, which can be further separated and refined. The crude DMT can then be distilled to obtain polymer grade material suitable for the preparation of polyesters that are similar to or the same as polyesters prepared from virgin materials.

Other methods for separating and purifying DMT and various glycol components from polyester depolymerization products have been described in, for example, U.S. Pat. Nos. 5,364,985; 5,391,263; 5,498,749; 5,712,410; 5,912,275 (Dupont); U.S. Pat. No. 6,706,843 (Teijin); U.S. Pat. Nos. 7,078,440; and 10,808,096, which are all incorporated herein by reference.

Briefly, in various embodiments, to facilitate methanol recovery, an azeotropic agent, such as methyl benzoate and/or methyl p-toluate, may be added to a mixture containing methanol and/or EG following the methanolysis reactor, to facilitate separating methanol and/or EG from DMT.

In case solid foreign materials are present in the reaction mixture, (A) a fraction of the solid foreign materials which has floated to the surface of the mixture, may be removed by means of a flotation separation method; (B) a fraction of the residual solid foreign materials which has not floated to the surface may be removed by a solid/liquid separation method; (C) the fraction from step (B) may be distilled and concentrated to recover a distilled EG; (D) the distillation residue from step (C) may be mixed with a transesterification reaction catalyst and methanol to cause a transesterification reaction between the distillation residue and methanol to occur and to produce DMT and EG, the reaction mixture may then be subjected to a recrystallization treatment and then to centrifugal separation to separate the reaction mixture into a DMT cake and a mixture solution, and the cake may be subjected to a distillation purification to recover the distilled DMT having a high degree of purity; (E) the mixture solution from step (D) may be subjected to a distillation treatment to recover the distilled methanol; and (F) the distillation residue from step (E) may be subjected to a distillation treatment to recover the distilled EG.

The foreign materials may comprise polyesters other than PET, polyvinyl chloride, polyvinylidene chloride, polyolefins, polystyrene, polyamides, polycarbonates, polyurethanes, polylactic acid, acryl, rayon, acetate, polyvinyl alcohol, natural plant fibers, natural animal fibers, metals, pigments, oils, inorganic compounds, sand, paper, wood, glass, asbestos, carbon black, dyes, and/or heat insulating materials.

The polyesters other than PET may comprise copolymerized PETs, polyethylene naphthalate, polytrimethylene terephthalate, and/or polybutylene terephthalate.

The polyolefins as foreign materials may comprise polyethylene and/or polypropylene.

The EG recovered in step (C) may be recirculated to step (A).

### Mixtures for Chemical Recycling of Waste Carpet Fibers

In another aspect, the invention provides a mixture for chemical recycling of waste carpet fibers. The mixture comprises the reaction product of:
(a) a waste carpet fiber composition comprising at least 75 wt % of polyethylene terephthalate (PET) and 6 wt % or less of ash; and
(b) methanol,
wherein the weight percentages are based on the total weight of the waste carpet fiber composition.

The waste carpet fiber composition in the mixture may have any of the features/parameters described herein.

The methanol may be in liquid or vapor form or both.

In one or more embodiments, the methanol may be a saturated vapor.

In one or more embodiments, the methanol may be superheated or supercritical.

In one or more embodiments, the methanol may be a superheated vapor.

The mixture may have a mass ratio of methanol to PET of 1.1:1 to 10:1.

The mixture may comprise other PET-containing waste products, such as textiles, bottle flake, reclaimer waste, or combinations thereof.

In one or more embodiments, the mixture comprises up to 95, up to 90, up to 85, up to 80, up to 75, up to 60, up to 50, up to 40, up to 30, up to 20, up to 10, up to 5, or up to 1 wt % of other PET-containing waste products, based on the total weight of the mixture.

The mixture may comprise an ester exchange catalyst.

Examples of ester exchange catalysts include zinc acetate, lithium acetate, manganese (II) acetate, titanium (IV) isopropoxide, magnesium methoxide, and potassium carbonate.

In one or more embodiments, the mixture comprises from 0 to 800 ppm, from 30 to 300 ppm, or from 30 to 100 ppm of an ester exchange catalyst, based on the total weight of the mixture.

The mixture may further comprise an ester exchange co-catalyst.

An example of an ester exchange co-catalyst includes sodium hydroxide.

In one or more embodiments, the mixture comprises from 0 to 800 ppm, from 30 to 300 ppm, or from 30 to 100 ppm of an ester exchange co-catalyst, based on the total weight of the mixture.

The mixture may further comprise dimethyl terephthalate, oligomers, or both.

The mixture may also comprise methoxy 2-hydroxyethyl terephthalate; bis(2-hydroxyethyl) terephthalate; diethylene glycol; dimethyl isophthalate; residual catalyst metals from PET, such as antimony, titanium, aluminum; dyes; colorants from the PET feed, inert solids, and/or dirt.

### Process of Preparing Recycled Polyester

In yet another aspect, the invention provides a process for preparing a recycled polyester. In one variation, the process comprises:
using the purified EG or DMT or both obtained according to the invention to prepare a recycled polyester.

In another variation, the process comprises:
reacting the purified DMT obtained according to the invention with water to form a recycled terephthalic acid (rTPA); and
using the rTPA and optionally the purified EG obtained according to the invention to prepare a recycled polyester.

In yet another variation, the process comprises:
reacting the purified DMT obtained according to the invention with virgin EG, the purified EG also obtained according to the invention, or both to form bis(2-hydroxyethyl) terephthalate (BHET) or oligomers thereof; and
polycondensing the BHET or the oligomers thereof to form a recycled PET.

Various processes are known for preparing polyesters from EG, DMT, or both. For example, DMT may be reacted with EG to produce an esterification product. The esterification product is then polycondensed at reduced pressure in the presence of a polycondensation catalyst to obtain PET.

### General Provisions

To remove any doubt, the present invention includes and expressly contemplates and discloses any and all combinations of embodiments, features, characteristics, parameters, and/or ranges mentioned herein. That is, the subject matter of the present invention may be defined by any combination of embodiments, features, characteristics, parameters, and/or ranges mentioned herein.

It is contemplated that any ingredient, component, or step that is not specifically named or identified as part of the present invention may be explicitly excluded.

Any process/method, apparatus, compound, composition, embodiment, or component of the present invention may be modified by the transitional terms “comprising,” “consisting essentially of,” or “consisting of,” or variations of those terms.

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein may be considered approximations. These values and ranges may vary from their stated numbers depending upon the desired properties sought to be obtained by the present disclosure as well as the variations resulting from the standard deviation found in the measuring techniques. Moreover, the ranges described herein are intended and specifically contemplated to include all sub-ranges and values within the stated ranges. For example, a range of 50 to 100 is intended to include all values within the range including sub-ranges such as 60 to 90, 70 to 80, etc.

Any two numbers of the same property or parameter reported in the working examples may define a range. Those numbers may be rounded off to the nearest thousandth, hundredth, tenth, whole number, ten, hundred, or thousand to define the range.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content.

This invention can be further illustrated by the following working examples, although these examples are included merely for purposes of illustration and are not intended to limit the scope of the invention.

### EXAMPLES

### Example 1

### Feedstocks

Various carpet fiber feedstocks were analyzed for PET content (calculated using IPA and TPA), ash content, 1,3-propanediol, nitrogen, and metals. The control sample (Control) was a high-grade, colored rPET flake that has been purified by a PET reclaimer and is suitable for mechanical recycling. The other samples were post-industrial (P-I) carpet fiber (Samples A, D, E, F), post-consumer (P-C) carpet fiber (Samples B, C, H-O), and a post-industrial carpet and carpet pad combination product (Sample G). For laboratory evaluation, all samples were densified either by melt extrusion or agglomeration.

To determine the TPA and IPA content, the feedstocks were analyzed by hydrolysis liquid chromatography, which is as described in [Anal. Chem. 1991, 63, 1251-1256]. This test has a margin of error of +/−3%. To calculate % PET and correct for the water added during the hydrolysis procedure, the sum of % TPA and % IPA (if measured) were divided by 0.864.If IPA was not measured, the value of % IPA was assumed to be zero.

To determine ash content, 1 g of sample was gravimetrically analyzed after being heated in air at 800° C. for 3 hours.

To quantitatively determine total nitrogen, samples were pulverized and measured by chemiluminescence using an NSX-2100H Trace Elemental Analyzer.

Propanediol was determined by hydrolysis gas chromatography. Samples are prepared for analysis by using a hydrolysis reaction and silylation. The sample solutions were chromatographed on a DB-5 column using a split injection and flame ionization detector. Weight precent concentrations of sample components were calculated from the integrated chromatograms using internal standard quantification.

Other metals, halogens, and non-metals were qualitatively determined by x-ray fluorescence on a Malvern/PANalytical Zetium XRF using the Omnian software package.

The results are shown in Table 1.

TABLE 1
Initial Feedstock Analysis of Carpet Samples

| | | | | | | Propane- | |
|---|---|---|---|---|---|---|---|
| | | PET | TPA | IPA | Ash | diol | Nitrogen |
| Sample | Description | (wt %) | (wt %) | (wt %) | (wt %) | (wt %) | (ppm) |
| Control | rPET | 101.3 | 84.61 | n/t | 0 | n/t | n/t |
| A | P-I - two | 100.7 | 84.05 | n/t | 0.18 | n/t | n/t |
| | tone dyed | | | | | | |
| B | P-C | 89.7 | 73.31 | 1.6 | 4.4 | n/t | n/t |
| C | P-C | 94.2 | 76.92 | 1.74 | 1.9 | n/t | 116 |
| D | P-C | 76.5 | 62.67 | 1.24 | 6.92 | n/t | n/t |
| E | P-I - undyed | 94.55 | 81.73 | n/t | 0.05 | n/t | 80 |
| F | P-I - dyed | 92.34 | 79.81 | n/t | 0.18 | n/t | 171 |
| G | P-I - PTT/PET | 97.7 | 79.75 | 1.79 | 0.14 | 13.2 | 42 |
| H | P-I with pad | 95.9 | 78.3 | 1.78 | 0.42 | 0 | 115 |
| I | P-C | 97.6 | 80.0 | 1.57 | 2.47 | n/t | 534 |
| J | P-C | 95.1 | 77.7 | 1.70 | 2.43 | n/t | 703 |
| K | P-C | 96.5 | 78.9 | 1.68 | 2.80 | n/t | 1326 |
| L | P-C | 96.3 | 78.9 | 1.52 | 2.12 | n/t | 1404 |
| M | P-C | 95.0 | 77.9 | 1.40 | 2.33 | n/t | 564 |
| N | P-C | 96.2 | 78.6 | 1.71 | 2.21 | n/t | 507 |
| O | P-C | 95.3 | 77.9 | 1.60 | 2.28 | n/t | 866 |
| P | P-C | 97.3 | 79.5 | 1.76 | 2.08 | n/t | 854 |

| | Al | Sb | Br | Ca | Cl |
|---|---|---|---|---|---|
| Sample | (wt %) | (wt %) | (wt %) | (wt %) | (wt %) |
| Control | 0 | 0.017 | 0.0003 | 0.001 | 0.0028 |
| A | 0 | 0.023 | 0.0003 | 0.001 | 0.0038 |
| B | 0.140 | 0.013 | 0.0023 | 1.57 | 0.0261 |
| C | 0.054 | 0.015 | 0 | 0.675 | 0.0213 |
| D | 0.413 | 0.0099 | 0 | 2.45 | 0.0514 |
| E | 0.413 | 0.010 | 0 | 2.45 | 0.0514 |
| F | 0 | 0.024 | 0.0006 | 0.002 | 0.0045 |
| G | 0 | 0.039 | 0.0559 | 0.005 | 0.0033 |
| H | 0.014 | 0.014 | 0 | 0.024 | 0.0072 |
| I | 0.048 | 0.027 | 0.0018 | 0.070 | 0.0120 |
| J | 0.060 | 0.026 | 0.0060 | 0.930 | 0.0350 |
| K | 0.073 | 0.025 | 0.0034 | 0.873 | 0.0213 |
| L | 0.048 | 0.024 | 0.0042 | 1.133 | 0.0313 |
| M | 0.048 | 0.023 | 0.0041 | 0.777 | 0.0267 |
| N | 0.065 | 0.025 | 0.0052 | 0.900 | 0.0303 |
| O | 0.064 | 0.027 | 0.0026 | 0.890 | 0.0257 |
| P | 0.057 | 0.024 | 0.0040 | 0.933 | 0.0253 |

| | Fe | Mg | Mn | K | Si | Na | Ti |
|---|---|---|---|---|---|---|---|
| Sample | (wt %) | (wt %) | (wt %) | (wt %) | (wt %) | (wt %) | (wt %) |
| Control | 0.0004 | 0 | 0 | 0 | 0 | 0 | 0.001 |
| A | 0.1020 | 0 | 0 | 0.0038 | 0 | 0 | 0.119 |
| B | 0.0433 | 0.349 | 0.0015 | 0.0153 | 0.0851 | 0 | 0.104 |
| C | 0.0359 | 0 | 0.0011 | 0.0119 | 0.0143 | 0 | 0.072 |
| D | 0.0557 | 0.269 | 0.0031 | 0.0209 | 0.0551 | 0 | 0.0743 |
| E | 0.0557 | 0.269 | 0.0031 | 0.0209 | 0.0551 | 0 | 0.074 |
| F | 0.0014 | 0 | 0 | 0.0058 | 0 | 0 | 0.211 |
| G | 0.0246 | 0 | 0 | 0.0065 | 0 | 0 | 0.313 |
| H | 0.0026 | 0.024 | 0 | 0 | 0.044 | 0 | 0.041 |
| I | 0.0050 | 0.069 | 0 | 0 | 0.037 | 0 | 0.150 |
| J | 0.0390 | 0.092 | 0 | 0.020 | 0.065 | 0.110 | 0.066 |
| K | 0.0427 | 0.096 | 0 | 0.019 | 0.059 | 0.078 | 0.073 |
| L | 0.0527 | 0.113 | 0 | 0.023 | 0.075 | 0.097 | 0.080 |
| M | 0.0353 | 0.087 | 0 | 0.021 | 0.058 | 0.099 | 0.055 |
| N | 0.0373 | 0.099 | 0 | 0.019 | 0.072 | 0.100 | 0.060 |
| O | 0.0447 | 0.099 | 0 | 0.017 | 0.064 | 0.092 | 0.078 |
| P | 0.0503 | 0.095 | 0 | 0.025 | 0.071 | 0.090 | 0.084 |

n/t = not tested

### Example 2

Laboratory Methanolysis Reaction and Purification

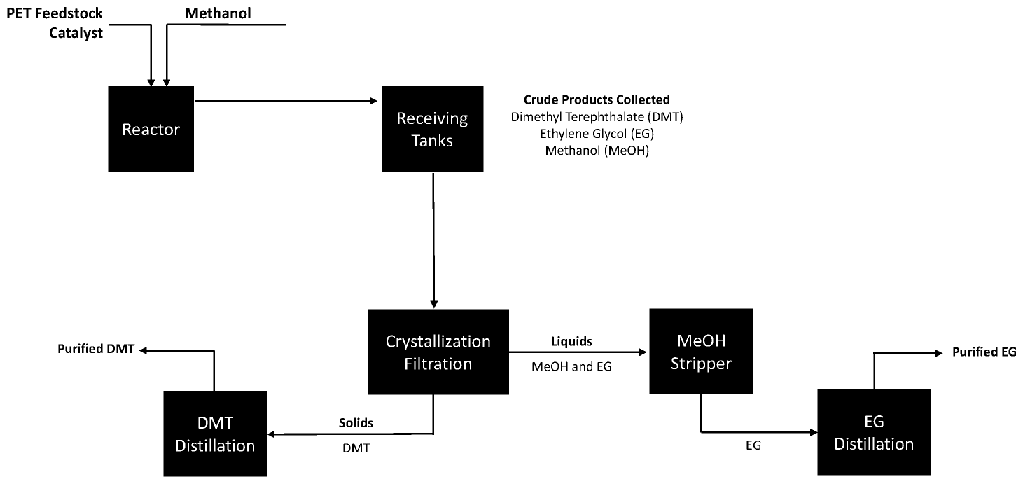

The samples from Example 1 were screened in a laboratory-scale methanolysis reactor and monomer purification system shown in FIG. 1.

Each methanolysis reaction was performed by adding an initial charge of PET carpet fiber feedstock, catalyst, and ethylene glycol to a 2-L reactor with dimensions of 11 inches (27.94 cm) deep and 4 inches (10.16 cm) in diameter. The ethylene glycol was added to help melt the PET feedstock in the batchwise laboratory scale reactor and was stripped out of the reactor within the first few hours of the procedure.

The reactor was heated to 260° C. to form a melt; then superheated methanol at 305° C. was sparged through the melt at a rate of 10 mL/min. The content level in the reactor was checked every hour. If the level fell below 7 inches (17.78 cm), then 100 g of the PET carpet fiber feedstock and the appropriate level of catalyst were added to the reactor. The initial charges and the charges for the subsequent additions were noted in 8-hour increments and reported Table 2. The crude products of DMT, EG, and MeOH were collected in receiving flasks/tanks for purification.

For purification, the products were crystallized in a stirred vessel, after which the crystallized products were filtered. The filter cake was then purified by batch column distillation to produce purified DMT.

Purified ethylene glycol can be produced by first removing methanol from the filtrate in a methanol stripping column, followed by purification via column distillation.

TABLE 2
Material Charges for Methanolysis Reactions

| | | | Initial Charge | | |
|---|---|---|---|---|---|
| | | Catalyst | (Elapsed Time = 0 hrs) | | |
| | | Loading | PET Material | Ethylene Glycol | Catalyst |
| Sample | Catalyst | (ppm of metal) | (g) | (g) | (g) |
| Control rPET | B | 150 | 1600 | 200 | 1.072 |
| Sample A | B | 150 | 1600 | 200 | 1.072 |
| 15 wt % Sample | A | 150 | 1600 | 200 | 0.800 |
| B in rPET | | | | | |
| 25 wt % Sample | A | 150 | 1600 | 200 | 0.800 |
| B in rPET | | | | | |
| 67 wt % Sample | A | 150 | 1600 | 200 | 0.801 |
| B in rPET | | | | | |
| Sample B | A | 150 | 1600 | 200 | 0.800 |
| Sample C | B | 150 | 1600 | 200 | 1.072 |
| Sample D | A | 150 | 1600 | 200 | 0.803 |

| | Charge 1 | | | Charge 2 | | |
|---|---|---|---|---|---|---|
| | (Elapsed Time = 8 hrs) | | | (Elapsed Time = 16 hrs) | | |
| | PET Material | Catalyst | MeOH | PET Material | Catalyst | MeOH |
| Sample | (g) | (g) | (mL) | (g) | (g) | (mL) |
| Control rPET | 400 | 0.269 | 3804.6 | 600 | 0.403 | 3404.6 |
| Sample A | 500 | 0.337 | 3804.6 | 600.5 | 0.408 | 3804.6 |
| 15 wt % Sample | 600.1 | 0.602 | 3804.6 | 400 | 0.4 | 3441.2 |
| B in rPET | | | | | | |
| 25 wt % Sample | 600 | 0.601 | 3804.6 | 600 | 0.601 | 3804.6 |
| B in rPET | | | | | | |
| 67 wt % Sample | 500.1 | 0.501 | 3804.6 | 600.1 | 0.601 | 3804.6 |
| B in rPET | | | | | | |
| Sample B | 600.2 | 0.603 | 3804.6 | 500.1 | 0.501 | 3804.6 |
| Sample C | 600.1 | 0.403 | 3804.6 | 700 | 0.47 | 3804.6 |
| Sample D | 400.2 | 0.399 | 3804.6 | 400 | 0.401 | 3804.6 |
| | Charge 3 | | | Charge 4 | | |
| | (Elapsed Time = 24 hrs) | | | (Elapsed Time = 32 hrs) | | |
| | PET Material | Catalyst | MeOH | PET Material | Catalyst | MeOH |
| Sample | (g) | (g) | (mL) | (g) | (g) | (mL) |
| Control rPET | 700 | 0.471 | 3804.6 | 700 | 0.47 | 3804.6 |
| Sample A | 700.4 | 0.47 | 3804.6 | 800 | 0.541 | 3804.6 |
| 15 wt % Sample | 800.1 | 0.803 | 3804.6 | 1100.1 | 1.102 | 3804.6 |
| B in rPET | | | | | | |
| 25 wt % Sample | 900 | 0.903 | 3804.6 | 900.1 | 0.902 | 3804.6 |
| B in rPET | | | | | | |
| 67 wt % Sample | 700.1 | 0.701 | 3804.6 | 800 | 0.802 | 3804.6 |
| B in rPET | | | | | | |
| Sample B | 600.7 | 0.604 | 3804.6 | 600.7 | 0.602 | 3804.6 |
| Sample C | 600.1 | 0.403 | 3804.6 | 700.1 | 0.47 | 3804.6 |
| Sample D | 300 | 0.303 | 3804.6 | 200.7 | 0.203 | 3804.6 |
| | Charge 5 | | | Charge 6 | | |
| | (Elapsed Time = 40 hrs) | | | (Elapsed Time = 48 hrs) | | |
| | PET Material | Catalyst | MeOH | PET Material | Catalyst | MeOH |
| Sample | (g) | (g) | (mL) | (g) | (g) | (mL) |
| Control rPET | 700.1 | 0.471 | 3804.6 | 800.1 | 0.538 | 3804.6 |
| Sample A | 701.4 | 0.471 | 3804.6 | 700.2 | 0.47 | 3804.6 |
| 15 wt % Sample | 600 | 0.6 | 3804.6 | 800.1 | 0.805 | 3804.6 |
| B in rPET | | | | | | |
| 25 wt % Sample | 600.1 | 0.601 | 3804.6 | 800.1 | 0.801 | 3804.6 |
| B in rPET | | | | | | |
| 67 wt % Sample | 700 | 0.701 | 3804.6 | 600 | 0.602 | 3804.6 |
| B in rPET | | | | | | |
| Sample B | 300.1 | 0.3 | 3804.6 | 601.1 | 0.604 | 3804.6 |
| Sample C | 800 | 0.537 | 3804.6 | 400 | 0.269 | 3804.6 |
| Sample D | 200.1 | 0.201 | 3804.6 | 300.3 | 0.301 | 3804.6 |
| | Charge 7 | | | Charge 8 | | |
| | (Elapsed Time = 56 hrs) | | | (Elapsed Time = 64 hrs) | | |
| | PET Material | Catalyst | MeOH | PET Material | Catalyst | MeOH |
| Sample | (g) | (g) | (mL) | (g) | (g) | (mL) |
| Control rPET | 700.1 | 0.47 | 3804.6 | 700.2 | 0.47 | 3804.6 |
| Sample A | 800.1 | 0.541 | 3804.6 | 700 | 0.473 | 3804.6 |
| 15 wt % Sample | 1000.1 | 1.002 | 3804.6 | 500.1 | 0.501 | 3804.6 |
| B in rPET | | | | | | |
| 25 wt % Sample | 900.1 | 0.902 | 3804.6 | 500.1 | 0.5 | 3804.6 |
| B in rPET | | | | | | |
| 67 wt % Sample | 600.1 | 0.601 | 2809.2 | datapoint not completed | | |
| B in rPET | | | | | | |
| Sample B | 404.2 | 0.502 | 3804.6 | 200.1 | 0.2 | 3804.6 |
| Sample C | 600 | 0.403 | 3804.6 | 500.1 | 0.336 | 3804.6 |
| Sample D | 300.1 | 0.303 | 3804.6 | 300.4 | 0.301 | 3804.6 |

Note:
Catalyst A = Zn(OAc)₂•2H₂O;
Catalyst B = Mn(OAc)₂•4H₂O

### Rate Data

The rate at which the reaction proceeded was assessed by calculating the molar ratio of MeOH/DMT for each timepoint and is shown in Table 3. Higher values indicate that more methanol was used to produce a mole of DMT and, therefore, are less desirable. The amount of methanol used was calculated from the rate of methanol addition and the time the methanol was being sparged through the reactor (Table 2). The amount of DMT produced was calculated from weighing the products in the receiving tanks and quantifying the concentration of DMT in the products using gas chromatography and liquid chromatography.

TABLE 3
Rate Data Based on Molar Ratio of
Methanol Consumed to DMT Produced

| | Elapsed Time (hr) | | | | | | | |
|---|---|---|---|---|---|---|---|---|
| Sample | 8 | 16 | 24 | 32 | 40 | 48 | 56 | 64 |
| Control | 10.21 | 5.85 | 6.23 | 5.57 | 6.05 | 5.20 | 5.38 | 5.85 |
| rPET | | | | | | | | |
| Sample A | 9.22 | 6.05 | 7.12 | 5.82 | 5.97 | 6.55 | 5.40 | 5.27 |
| 15 wt % | 8.37 | 5.10 | 5.68 | 5.57 | 4.69 | 5.26 | 5.38 | 4.59 |
| Sample | | | | | | | | |
| B in rPET | | | | | | | | |
| 25 wt % | 8.62 | 4.41 | 5.06 | 5.75 | 5.80 | 4.50 | 5.63 | 5.66 |
| Sample | | | | | | | | |
| B in rPET | | | | | | | | |
| 67 wt % | 9.72 | 6.19 | 11.53 | 6.05 | 6.38 | 7.73 | 7.12 | — |
| Sample | | | | | | | | |
| B in rPET | | | | | | | | |
| Sample B | 16.46 | 8.03 | 9.62 | 9.03 | 10.59 | 11.42 | 13.41 | 12.34 |
| Sample C | 10.62 | 6.07 | 6.57 | 5.84 | 6.42 | 6.33 | 6.74 | 8.29 |
| Sample D | 25.30 | 16.40 | 18.80 | 19.11 | 26.69 | 30.18 | 33.27 | 32.62 |

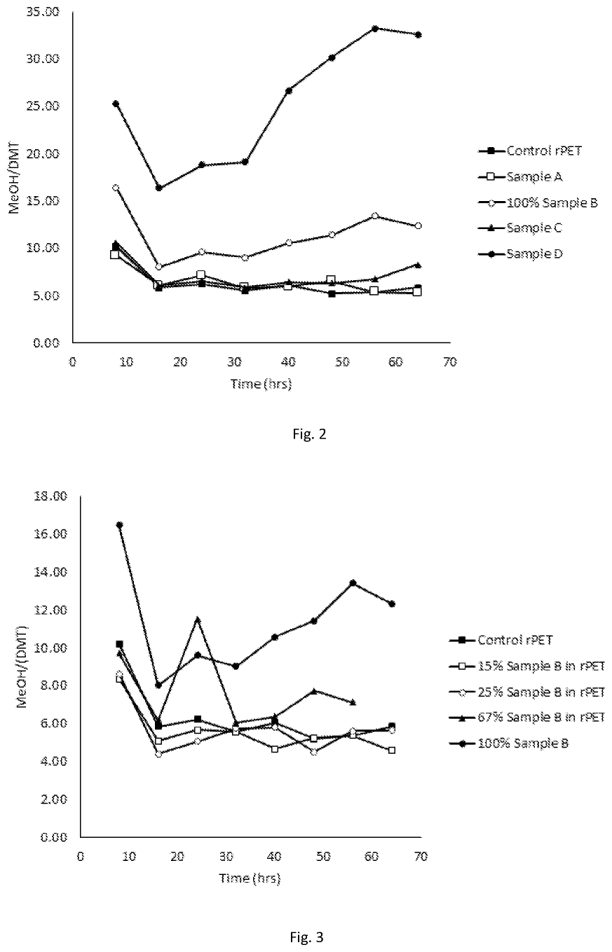

The rate data in Table 3 are depicted graphically in FIGS. 2 and 3. MeOH/DMT was used as a proxy for the rate of reaction, where lower values of MeOH/DMT indicate fewer moles of MeOH were needed to produce one mole of DMT.

As seen in FIG. 2, a higher PET content in a feedstock correlated to a lower MeOH/DMT value. For some feedstocks, particularly, those containing post-consumer carpet fibers, a lower PET content was related to a higher ash content due to the way the samples were prepared, which correlated to higher MeOH/DMT values. For example, when comparing the three post-consumer samples (B, C and D), sample D had the lowest PET % (76.5 wt %) and the highest Ash % (6.92 wt %) and proceeded at the highest MeOH/DMT values. Sample B had the intermediate level of PET % and Ash % (89.7 wt % and 4.4 wt %, respectively) and proceeded at an intermediate MeOH/DMT rate. Sample C had the highest PET % (94.2 wt %) and the lowest Ash % (1.9 wt %) and proceeded at the lowest MeOH/DMT values. Feedstocks with higher ash contents also tended to show a degradation in rate over time as the inorganic content filled the reactor with inactive material.

Sample A, the post-industrial carpet fiber sample, had the lowest level of Ash % (0.18 wt %) and the highest PET % (100.7 wt %). This feedstock had very similar PET % and Ash % to the Control sample and, therefore, had a very similar rate.

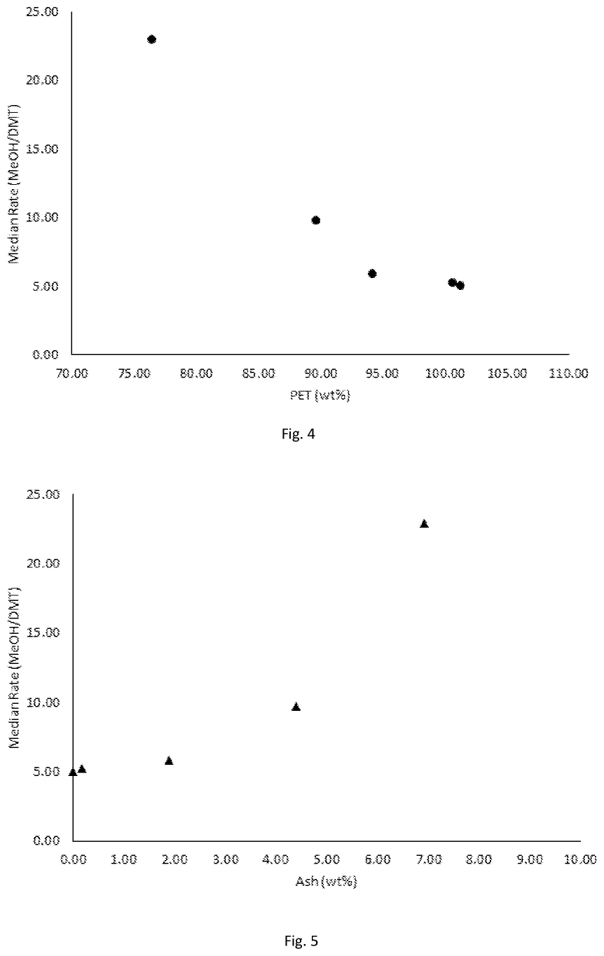

FIGS. 4 and 5 are graphs comparing the median MeOH/DMT values of the Control and A-D samples with the feedstock characteristics, PET % and Ash %, respectively. As seen from these graphs, the MeOH/DMT values were surprisingly not a linear function of either the PET % or the Ash %. The median MeOH/DMT values were calculated using the results from the elapsed timepoints 24-64 hrs (omitting the elapsed timepoints 8 and 16 hrs). The early timepoints, 8 and 16 hrs, often showed noise from a lack of equilibrium in the reaction.

FIG. 3 shows the rate data for the Control sample, sample B, and various mixtures of the two. The trends observed in FIG. 2 carried over to FIG. 3.

Moreover, as seen in FIG. 3, the sample diluted to 15% surprisingly performed about the same as the sample that was diluted to 25%. These results suggest a non-linear response to PET % in the carpet fiber feedstock. Additionally, it was surprising to find that in general, pure DMT could be obtained from a carpet fiber sample containing all of the impurities listed above.

### Purified DMT

As noted above, after the DMT from each carpet sample was isolated from the reaction in the receiving flasks, it was purified by crystallization, filtration, and distillation. The impurities in the DMT after distillation are reported in Table 4. The overall assay of the DMT was determined by gas chromatography, and the metals present were detected by X-ray fluorescence. Some of the samples were collected in two cuts from the distillation.

For comparison, a virgin, commercially-produced DMT (labeled as Distilled DMT) was redistilled in the same manner as the methanolysis feedstocks. Its impurities after distillation are also reported in Table 4.

In Table 4, MHET is methylhydroxyethyl terephthalate, MHT is monohydroxyethyl terephthalate, BHET is bis(hydroxyethyl) terephthalate, and DMI is dimethyl isophthalate.

TABLE 4
Purified DMT

| | Gas Chromatography Analysis (wt %) | | | | | | |
|---|---|---|---|---|---|---|---|
| Sample | MHET | MHT | BHET | DMI | Water | DMT | Unknowns |
| Control - Distilled | 0 | 0 | 0.02 | 0 | 0.74 | 99.95 | 0.08 |
| DMT, Cut #1 | | | | | | | |
| Control - Distilled | 0 | 0 | 0.03 | 0 | 0.8 | 99.36 | 0 |
| DMT, Cut #2 | | | | | | | |
| Control Sample, | 0 | 0 | 0 | 0 | 0.31 | 97.02 | 2.05 |
| cut #1 | | | | | | | |
| Control Sample, | 0 | 0 | 0 | 0 | 0.77 | 96.94 | 0.48 |
| cut #2 | | | | | | | |
| Sample A, cut #1 | 0 | 0 | 0 | 0.1 | 0.95 | 98.77 | 0.13 |
| Sample A, cut #2 | 0 | 0 | 0 | 0.08 | 0.81 | 100.43 | 0.09 |
| Sample B, cut #1 | 0 | 0 | 0 | 0 | 1.09 | 100.94 | 0.1 |
| Sample B, cut #2 | 0 | 0 | 0 | 0 | 0.8 | 101.11 | 0.04 |

| | X-Ray Fluoresence (ppm) | | | | |
|---|---|---|---|---|---|
| Sample | Ti | Si | Sb | Cl | Br |
| Control - Distilled | BDL | BDL | BDL | BDL | BDL |
| DMT, Cut #1 | | | | | |
| Control - Distilled | BDL | BDL | BDL | BDL | BDL |
| DMT, Cut #2 | | | | | |
| Control Sample, | 1.8 | BDL | BDL | 9.6 | BDL |
| cut #1 | | | | | |
| Control Sample, | 2.1 | BDL | BDL | 11 | BDL |
| cut #2 | | | | | |
| Sample A, cut #1 | BDL | BDL | BDL | BDL | BDL |
| Sample A, cut #2 | BDL | BDL | BDL | BDL | BDL |
| Sample B, cut #1 | 2.4 | BDL | BDL | 0.5 | 1.8 |
| Sample B, cut #2 | 1.6 | BDL | BDL | 13 | BDL |

BDL = Below Detection Limit

### Example 3

### Copolyester Synthesis

Some of the purified, recycled DMT (rDMT) from Example 2 were screened for fitness-for-use in copolyester production by synthesizing an amorphous copolyester containing 2,2,4,4-tetramethyl-1,3-cyclobutenediol (TMCD) and 1,4-cyclohexanedimethanol (CHDM). All copolyesters were produced using the second cut from the batch distillation described above. rDMT (77.68 g), CHDM (38.05 g), and TMCD/MeOH Solution (35 wt % TMCD, 67.11 g solution) were weighed into a 500-mL single-neck flask. To the monomers, a catalyst solution in n-butanol containing a phosphorus compound and a tin compound was added to target a final catalyst concentration of 125 ppm Sn and 8 ppm P. The flask was equipped with a motorized stirring system, a side-arm condenser, a condensate receiving flask, a dry ice-acetone trap, and a manifold to achieve inert (N₂) and vacuum atmosphere. Heating of the flask was achieved by lowering the flask into a molten metal bath in contact with a heating mantle. An automation program was used to control the temperatures, pressures, and stirring rates throughout the reaction.

Under N₂ at atmospheric pressure, the flask was gradually heated from 220° C. to 245° C. over 25 minutes, and then held at 245° C. for 40 minutes. The pressure was then reduced to 250 torr and the pressure was increased to 265° C. over 18 minutes. The pressure was then reduced to 1.5 torr and the temperature was increased to 277° C. over 8 minutes, and then held at those conditions for 37 minutes. After terminating this sequence, the flask was returned to atmospheric conditions and the polymer was removed for analysis.

The properties of the resulting copolyesters were measured and are reported in Table 5.

TABLE 5
Polymer Properties

DMT Source

| | Control - | rPET | | |
|---|---|---|---|---|
| Copolyester | Distilled | Control | | |
| Properties | DMT | Sample | Sample A | Sample B |
| Inherent | 0.65 | 0.64 | 0.64 | 0.65 |
| Viscosity | | | | |
| (dL/g) | | | | |
| Methyl Ends | 1.55 | 2.03 | 1.95 | 1.92 |
| (%) | | | | |
| Carboxyl | 3.00 | 3.73 | 3.46 | 3.6 |
| End Groups | | | | |
| (meq/kg) | | | | |
| Base | 1.136 | 1.314 | 1.720 | 1.077 |
| Yellowness | | | | |
| (PLQ) | | | | |
| Haze | 1.51 | 2.02 | 4.22 | 2.9 |
| (%, PLQ) | | | | |

In Table 5, the rPET Control, Sample A (post-industrial carpet fiber), and Sample B (post-consumer carpet fiber) polymers were synthesized from rDMT produced via laboratory methanolysis and purification. As noted above, the Distilled DMT Control was virgin, commercially produced DMT that has been redistilled in the same manner as the methanolysis feedstocks.

As seen in Table 5, all of the polymers had similar IVs and end-group compositions, indicating that the rDMT samples produced by methanolysis were of high enough purity to produce a commercial-grade copolyester. Base Yellowness and Haze were measured because oftentimes immeasurably small concentrations of impurities can cause quality problems, so these values are an indication of the capability for the rDMT to make high quality copolyesters. Both the rPET Control and Samples A and B polymers were within a reasonable range of the Distilled DMT Control polymer, so that the rDMT obtained by methanolysis of waste PET carpet fibers can be considered for commercial production of high quality copolyesters.

The invention has been described in detail with particular reference to specific embodiments thereof, but it will be understood that variations and modifications can be made within the spirit and scope of the invention.

One aspect of the present invention is a mixture for chemical recycling of waste carpet fibers, the mixture comprising the reaction product of:
(a) a waste carpet fiber composition comprising at least 75 wt % of polyethylene terephthalate (PET) and 6 wt % or less of ash; and
(b) methanol,
wherein the weight percentages are based on the total weight of the waste carpet fiber composition.

One embodiment of this aspect is wherein the waste carpet fiber composition comprises at least 90 wt %, at least 95 wt %, or 100 wt % of PET.

One embodiment of this aspect and the previous embodiment is wherein the waste carpet fiber composition comprises 3 wt % or less, 1 wt % or less, 0.5 wt % or less, or 0 wt % of ash.

One embodiment of this aspect and the previous embodiments is wherein the waste carpet fiber composition comprises greater than 0 up to 6 wt %, greater than 0 up to 3 wt %, greater than 0 up to 1 wt %, or greater than 0 up to 0.5 wt % of ash.

One embodiment of this aspect and the previous embodiments is wherein the waste carpet fiber composition comprises post-industrial carpet fibers, post-consumer carpet fibers, or both.

One embodiment of this aspect and the previous embodiments is wherein the waste carpet fiber composition comprises at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-consumer carpet fibers, based on the total weight of the waste carpet fiber composition.

One embodiment of this aspect and the previous embodiments is wherein the waste carpet fiber composition comprises at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-industrial carpet fibers, based on the total weight of the waste carpet fiber composition.

One embodiment of this aspect and the previous embodiments is wherein the waste carpet fiber composition comprises up to 10 wt %, up to 3 wt %, or 0 wt % of the residues of isophthalic acid, based on the total weight of the waste carpet fiber composition.

One embodiment of this aspect and the previous embodiments is wherein the waste carpet fiber composition comprises up to 15 wt %, up to 5 wt %, or 0 wt % of the residues of propanediol, based on the total weight of the waste carpet fiber composition.

One embodiment of this aspect and the previous embodiments is wherein the waste carpet fiber composition comprises up to 5000 ppm, up to 1500 ppm, up to 200 ppm, up to 80 ppm, or 0 ppm of nitrogen, based on the total weight of the waste carpet fiber composition.

One embodiment of this aspect and the previous embodiments is wherein the waste carpet fiber composition comprises densified waste carpet fibers.

One embodiment of this aspect and the previous embodiments further comprises an ester exchange catalyst and optionally, an ester exchange co-catalyst.

One embodiment of this aspect and the previous embodiments further comprises dimethyl terephthalate, oligomers, or both.

One embodiment of this aspect and the previous embodiments further comprises the reaction product of one or more other PET-containing waste products and methanol.

One embodiment of this aspect and the previous embodiments is wherein the other PET-containing waste products comprise textiles, bottle flake, reclaimer waste, or combinations thereof.

1. A process for chemically recycling waste polyethylene terephthalate (PET) carpet fibers, the process comprising:
providing a waste carpet fiber composition comprising at least 75 wt % of PET and 6 wt % or less of ash;
reacting the waste carpet fiber composition with methanol to produce a depolymerized polyester mixture comprising polyester oligomers, dimethyl terephthalate (DMT), and ethylene glycol (EG); and
recovering the DMT and the EG from the depolymerized polyester mixture, wherein the weight percentages are based on the total weight of the waste carpet fiber composition.

2. The process according to claim 1, wherein the waste carpet fiber composition comprises at least 80 wt %, at least 85 wt %, at least 90 wt %, at least 95 wt %, or 100 wt % of PET.

3. The process according to claim 1, wherein the waste carpet fiber composition comprises 3 wt % or less, 1 wt % or less, 0.5 wt % or less, or 0 wt % of ash.

4. The process according to claim 1, wherein the waste carpet fiber composition comprises greater than 0 up to 6 wt %, greater than 0 up to 3 wt %, greater than 0 up to 1 wt %, or greater than 0 up to 0.5 wt % of ash.

5. The process according to claim 1, wherein the waste carpet fiber composition comprises post-industrial carpet fibers, post-consumer carpet fibers, or both.

6. The process according to claim 1, wherein the waste carpet fiber composition comprises at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-consumer carpet fibers, based on the total weight of the waste carpet fiber composition.

7. The process according to claim 1, wherein the waste carpet fiber composition comprises at least 5 wt %, at least 10, wt % at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, or at least 90 wt % and/or not more than 100 wt %, not more than 99 wt %, not more than 95 wt %, not more than 90 wt %, not more than 85 wt %, not more than 80 wt %, not more than 75 wt %, not more than 70 wt %, not more than 65 wt %, not more than 60 wt %, not more than 55 wt %, not more than 50 wt %, not more than 45 wt %, not more than 40 wt %, or not more than 35 wt % of post-industrial carpet fibers, based on the total weight of the waste carpet fiber composition.

8. The process according to claim 1, wherein the waste carpet fiber composition comprises up to 10 wt %, up to 3 wt %, or 0 wt % of the residues of isophthalic acid, based on the total weight of the waste carpet fiber composition.

9. The process according to claim 1, wherein the waste carpet fiber composition comprises up to 15 wt %, up to 5 wt %, or 0 wt % of the residues of 1,3-propanediol, based on the total weight of the waste carpet fiber composition.

10. The process according to claim 1, wherein the waste carpet fiber composition comprises up to 5000 ppm, up to 1500 ppm, up to 200 ppm, up to 80 ppm, or 0 ppm of nitrogen, based on the total weight of the waste carpet fiber composition.

11. The process according to claim 1, wherein the waste carpet fiber composition comprises densified waste carpet fibers.

12. The process according to claim 1 wherein the reacting step is conducted in the presence of an ester exchange catalyst and optionally, an ester exchange co-catalyst.

13. The process according to claim 1, wherein the methanol is superheated or supercritical.

14. The process according to claim 1, wherein the molar ratio of methanol consumed to DMT generated throughout the reacting step is 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, or 5 or less.

15. The process according to claim 1, wherein the DMT, the EG, or both are purified by crystallization, filtration, distillation, or combinations thereof.

16. The process according to claim 1, which produces virgin-quality EG, virgin-quality DMT, or both.

17. The process according to claim 1, wherein the reacting step is conducted in the presence of one or more other PET-containing waste products.

18. The process according to claim 17, wherein the other PET-containing waste products comprise textiles, bottle flake, reclaimer waste, or combinations thereof.

19. A process for preparing a recycled polyester, the process comprising:
using the purified EG or DMT or both from the process of claim 15 to prepare a recycled polyester; or
reacting the purified DMT from the process of claim 15 with water to form a recycled terephthalic acid (rTPA); and
using the rTPA and optionally the purified EG to prepare a recycled polyester.

20. A process for preparing recycled polyethylene terephthalate (rPET), the process comprising:
reacting the purified DMT from the process of claim 15 with virgin EG, the purified EG, or both to form bis(2-hydroxyethyl) terephthalate (BHET) or oligomers thereof; and
polycondensing the BHET or the oligomers thereof to form the rPET.
